(19) United States
(12) Patent Application Publication
(10) Pub. No.: US 2008/0262019 A1
(43) Pub. Date: Oct. 23, 2008

(54) Optical Isomers of (+) and (-)-Trans-2,3,4,4A,5,9B-Hexahydro-2,8-Dimethyl-1H-Pyrido[4,3-B] Indole

(76) Inventors: Ramiz Medzhidovich Salimov, Moscow (RU); Georgy Ivanovich Kovalev, Moscow (RU); Maria Nikolaevna Preobrazhenskaya, Moscow (RU); Sergey Nikolaevich Lavrenov, Moscow (RU); Sergey Alexandrovich Lakatosh, Moscow (RU)

Correspondence Address: MORGAN & FINNEGAN, L.L.P., 3 WORLD FINANCIAL CENTER, NEW YORK, NY, 10281-2101, US

(21) Appl. No.: 11/816,129

(22) PCT Filed: Sep. 12, 2005
(86) PCT No.: PCT/RU05/00459

§ 371 Date: Aug. 13, 2007

(51) Int. Cl. A61K 31/437; C07D 471/04; A61P 25/00

(52) U.S. Cl. 514/292; 546/85

(57) ABSTRACT

The present invention represents novel optical isomers produced according the invention that are optic antipodes to each other having individual biological activity which can be used as active ingredients in pharmaceutical compositions with nootropic and sedative activity for treatment of different individual conditions of patients.

### TECHNICAL FIELD

The present invention relates to new compounds, namely, to optical isomers of trans-2,3,4,4a,5,9b-hexahydro-2,8-dimethyl-1H-pyrido[4,3-b]indole, ways of their production and use on the basis of revealed nootropic and sedative therapeutic action.

### PREVIOUS TECHNICAL LEVEL

Known are derivatives of hexa-gamma-carboline 8-alkyl-2-[gamma-(p-fluorobenzoil)-propyl]-2,3,4,4a,5,9b-hexahydro-1H-pyrido[4,3-b]indoles and their pharmaceutically acceptable salts (U.S. Pat. No. 3,983,239 B1), with depressant effect on the central nervous system, capable of diminishing the stereotype behavior induced by amphetamine and being neuroleptics.

Known is 3,6-dimethyl-1,2,3,4,4a,9a-hexahydro-γ-carboline dihydrochloride, with pharmacological activity and a drug on its basis having international non patented name “Dicarbin” (U.S. Pat. No. 3,657,254 B2). Psychotropic activity of Dicarbine is described, including its neuroleptic, antidepressant and activating effects, that are observed only depending upon the symptoms of the disease and the state of the patient, and the therapeutical effect depends on the dose and the duration of the administration.

Known is way of synthesis of Dicarbine (U.S. Pat. No. 3,657,254 B1), that includes reduction of 3,6-dimethyl-1,2,3,4,4a,9a-hexahydro-γ-carboline hydrochloride in water acidic media at temperatures up to 100° C., following increase in the pH of the media by bases and separation of the resulting base of the 3,6-dimethyl-1,2,3,4,4a,9a-hexahydro-γ-carboline, treatment of this base by hydrochloric acid and purification of the target product, in which as reducers it is desirable to use amalgams of zink or tin. Using this method the cis-isomer of Dicarbin is obtained where hydrogen atoms in 4a and 9b positions are connected to carbon atoms in cis-position.

Known that 2,8-dimethyl-2,3,4,4a,5,9b-hexahydro-1H-pyrido[4,3-b]indole, that has pharmacological name Dicarbine, is acting as a depressant of the central nervous system and in parallel has a bi-phasic activity with regards to the effects of Amphetamine and antagonism to the stereotype behavior induced by Amphetamine (Psychopharmacologia, Vol. 21, 1971, pp. 82-88).

Known is pharmacological activity, specifically antiarrhythmic and antioxidant, for pyridoindole Stobadine, that is an optical isomer of cis-Dicarbine (L. Horakova and S. Stole. “Antioxidant Pharmacodynamic Effects of Pyridoindole Stobadine”. General Pharm., vol. 30, Issue 5, pp. 627-638)

Known is a selective method of synthesis of racemic cis-Dicarbine by catalytic hydration of the corresponding γ-carboline (L. Benes, S. Stolc. Drugs of the Future. 1989, Vol. 14, No. 2, pp. 135-137). Next, the optical isomers of cis-Dicarbine were obtained from the racemic mixture by crystallizing its salts with (+) and (−) dibenzoylvynil acids.

Known is racemic trans-2,3,4,4a,5,9b-hexahydro-1H-pyrido[4,3-b]indole hydroclorides, that has hydrogen atoms at 4a and 9b in trans-position and used as an analgesic and sedative drug. It is also used as a major tranquilizer with antipsychotic activity, or as a minor tranquilizer with anxiolythic activity, relaxant and hypotensive drug (U.S. Pat. No. 3,991,199 B2).

Known is a method of synthesis of 2,8-dimethyl-2,3,4,4a,5,9b-hexahydro-1H-pyrido[4,3-b]indole (2,8-dimethyl-γ-carboline) (Yahontov, L. N., Glushkov, R. G. Modem medicines. Meditsina, 1983, pp. 234-237) by reduction of 2,8-dimethyl-γ-carboline by zink dust. This method yields a racemic mixture of cis- and trans-isomers of Dicarbine with more cis-isomer.

Known is a method of selective synthesis of the racemic trans 2,8-dimethyl-2,3,4,4a,5,9b-hexahydro-1H-pyrido[4,3-b]indole by hydroborating the 2,8-dimethyl-γ-carboline by BF₃.Et₂O complex (Y. Nagai et al., Journal of Medicinal Chemistry. 1979, Vol. 22, N. 6, pp. 677-683).

However, all active compounds mentioned above are the racemic mixtures of optical isomers with varying therapeutic activities. This can result in undesirable side effects, specifically during a combined therapy by cognition-enhancers and sedative drugs, which dosage has to be precise.

Known methods of obtaining the mentioned active compounds do not allow for separation of the individual optical isomers from the racemic mixture of trans-isomer. Unique pharmacological activity of (+)- and (−)-isomers of trans-2,3,4,4a,5,9b-hexahydro-2,8-dimethyl-1H-pyrido[4,3-b]indole, that is a combination of multiple therapeutic effects, cannot be observed.

### DISCLOSURE OF INVENTION

The objective of the invention is to obtain individual substances from the racemic mixture that demonstrate individual therapeutic activity.

The objective was to separate the isomers of trans-2,8-dimethyl-2,3,4,4a,5,9b-hexahydro-1H-pyrido[4,3-b]indole that is used as analgesic and sedative drug. These isomers have hydrogen atoms 4a and 9b in trans-position. Individual (+)- and (−)-optical isomers of the trans-2,8-dimethyl-2,3,4,4a,5,9b-hexahydro-1H-pyrido[4,3-b]indole were obtained.

Besides, the objective was to test individual therapeutic activity of the mentioned above optical isomers in order to evaluate their options for clinical use.

The objective was achieved by developing a method of obtaining individual optical isomers (+)-trans-2,8-dimethyl-2,3,4,4a,5,9b-hexahydro-1H-pyrido[4,3-b]indole and (−)-trans-2,8-dimethyl-2,3,4,4a,5,9b-hexahydro-1H-pyrido[4,3-b]indole according to the invention, that utilizes interaction of 2,8-dimethyl-γ-carboline with sodium borohydrate and with etherate of trifluorine boron, treatment with hydrochloric acid, then alkilinization of the reaction mixture, separation of the trans-2,8-dimethyl-2,3,4,4a,5,9b-hexahydro-1H-pyrido[4,3-b]indole free base racemate, crystallization of its salts with (+)-dibenzoyltartaric acid from ethanol resulting in obtaining individual salt of the (+)-trans isomer. From individual salt the (+)-trans-2,8-dimethyl-2,3,4,4a,5,9b-hexahydro-1H-pyrido[4,3-b]indole free base was obtained, the remainder was converted into the free racemic base of the trans-2,8-dimethyl-2,3,4,4a,5,9b-hexahydro-1H-pyrido[4,3-b]indole. This base is crystallized with (−)-dibenzoyltartaric acid from ethanol resulting in obtaining individual salt of the (−)-trans isomer and later the (−)-trans-2,8-dimethyl-2,3,4,4a,5,9b-hexahydro-1H-pyrido[4,3-b]indole free base was obtained.

The objective was achieved by obtaining a new compound, that is an optical isomer (+)-trans-2,8-dimethyl-2,3,4,4a,5,9b-hexahydro-1H-pyrido[4,3-b]indole with positive optical rotation, where hydrogen atoms in positions 4a and 9b are in trans-position and has a structural formula of:

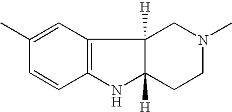

The objective was also achieved by obtaining a new compound, that is an optical isomer (−)-trans-2,3,4,4a,5,9b-hexahydro-2,8-dimethyl-1H-pyrido[4,3-b]indole with negative optical rotation, where hydrogen atoms in positions 4a and 9b are in trans-position and has a structural formula of:

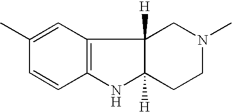

The objective was also achieved by developing a pharmaceutical composition with nootropic and sedative activity that includes optical isomer (+)-trans-2,3,4,4a,5,9b-hexahydro-2,8-dimethyl-1H-pyrido[4,3-b]indole and/or its pharmacologically acceptable salt in effective amount.

The objective was also achieved by developing a pharmaceutical composition with nootropic and sedative activity that includes optical isomer (−)-trans-2,3,4,4a,5,9b-hexahydro-2,8-dimethyl-1H-pyrido[4,3-b]indole and/or its pharmacologically acceptable salt in effective amount.

The invention is further illustrated by the examples of its realization that are however not limiting possibilities of its realization.

### The Best Way of the Invention Realization

A. Obtaining (+)-trans-2,8-dimethyl-2,3,4,4a,5,9b-hexahydro-1H-pyrido[4,3-b]indole and (−)-trans-2,8-dimethyl-2,3,4,4a,5,9b-hexahydro-1H-pyrido[4,3-b]indole optic isomers. The isomers were obtained according to invention in two stages by the following process:
1. Obtaining racemic isomer of trans-2,8-dimethyl-2,3,4,4a,5,9b-hexahydro-1H-pyrido[4,3-b]indole.

The racemic isomer of trans-2,8-dimethyl-2,3,4,4a,5,9b-hexahydro-1H-pyrido[4,3-b]indole is known to contain two optical isomers with hydrogen atoms in 4a and 9b positions in trans-position and the rotational angles are opposite—positive rotation for (+)-trans-isomer and negative rotation for (−)-trans-isomer:

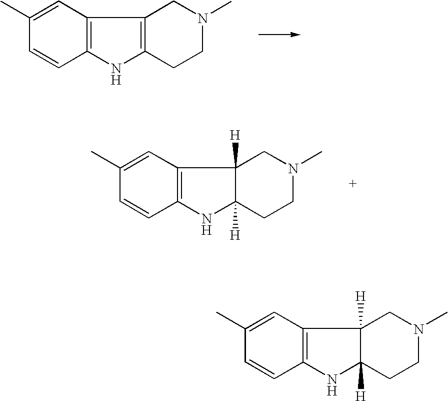

2,8-dimethyl-γ carboline (20 g, 0.1 mol) solution in tetrahydrofurane (THF)(300 ml) on ice was treated by a fine powdered sodium borohydrate (7.6 g, 0.2 mol), followed by slow (within an hour) addition of triftorborate ether (48% BF₃) (38 g, 0.27 mol) solution in THF on high stirring in an inert gas atmosphere. After adding all BF₃ the reaction mix was stirred for 30 min at room temperature and then boiled for 4 hours.

After cooling of the reaction mix the 6N-solution of HCl (150 ml) was added and the mixture was evaporated under THF vacuum. Resulting mixture was mixed with dioxane (150 ml), boiled for 1 hour and then evaporated. Next the resulting mixture was treated by NaOH solution and extracted by CHCl₃.

According to TLC, the resulting compound was a free base of trans-2,8-dimethyl-2,3,4,4a,5,9b-hexahydro-1H-pyrido[4,3-b]indole, containing less than 10% of the cis-isomer, which was removed by crystallization. In order to remove the cis-isomer resulting mixture was transformed into the mixture of dihydrochlorides of the cis- and trans-isomers by treating it with the ethanol solution of HCl. Following crystallization of trans-isomer hydrochloride from ethanol allowed for removal of the cis-isomer from the mixture. The alkalinization of racemic trans-isomer dihydrochloride and subsequent extraction resulted in obtaining an individual racemic base of trans-2,8-dimethyl-2,3,4,4a,5,9b-hexahydro-1H-pyrido[4,3-b]indole.

The resulting product was evaluated by TLC and NMR. Analytical TLC was performed using Kieselgel 60 F₂₅₄ plates (Merck).

According to results of the testing the obtained compounds:
for the racemic trans-isomer R_{f}=0.33 (in petroleum ether-ethylacetate-triethylamine system, 3:1:0.1);
for cis-isomer (Dicarbine) R_{f}=0.39 (in the same system).

According to TLC and NMR data obtained cis-isomer was identical to the known sample of the cis-isomer, obtained by the published method (Yahontov, L N, Glushkov, R G, Synthetic medicines. Medicina, 1983, pp. 234-237).

The yield of the base of trans-isomer was around 70% and it was used to obtain individual optical isomers.

For resulting products:

A) The melting points were measured on Buchi SMP-20 and were not corrected:
the melting point Tₘ of the racemic trans-2,8-dimethyl-2,3,4,4a,5,9b-hexahydro-1H-pyrido[4,3-b]indole dihydrochloride was 258-260° C.;
the melting point Tₘ of the trans-2,8-dimethyl-2,3,4,4a,5,9b-hexahydro-1H-pyrido[4,3-b]indole free base was 83-87° C.
   B) The chromatographic mobility (analytical TLC was performed using Kieselgel 60 F₂₅₄ plates (Merck)):
for trans-2,8-dimethyl-2,3,4,4a,5,9b-hexahydro-1H-pyrido[4,3-b]indole the R_{f} 0.43 (in the chloroform:methanol:triethylamine system, 10:1:0.1);
for cis-2,8-dimethyl-2,3,4,4a,5,9b-hexahydro-1H-pyrido[4,3-b]indole (Dicarbine) in the same system R_{f} 0.35.
   2. Separation of the racemic isomer of the trans-2,8-dimethyl-2,3,4,4a,5,9b-hexahydro 1H-pyrido[4,3-b]indole into optical isomers (+)- and (−)-trans-2,8-dimethyl-2,3,4,4a,5,9b-hexahydro-1H-pyrido[4,3-b]indole.

The 10 g trans-2,8-dimethyl-2,3,4,4a,5,9b-hexahydro-1H-pyrido[4,3-b]indole racemic base solution in 150 ml ethanol was mixed with the equimolar amount of the (+)-dibenzoyltartaric acid (18.61 g). After some time the crystals of the salt enriched in (+)-trans-isomer precipitate.

The precipitate was several times re-crystallized from ethanol to achieve the resulting angle of rotation for free base [α]_{D}²⁰+58° (Cl, CHCl₃), for hydrochloride [α]²⁰_{D}: +7° (Cl, H₂O), as measured by Perkin-Elmer 241 polarimeter. Free base of the (+)-isomer was obtained by alkilinisation of the salt solution by adding NaOH with subsequent extraction by ether and evaporation.

Next, after the removal of the (+)-isomer salt, the matrix solution was treated by the alkali solution, free base was removed by ether extraction and after evaporation under vacuum the resulting mixture contained bases, enriched in (−)-trans-2,8-dimethyl-2,3,4,4a,5,9b-hexahydro-1H-pyrido[4,3-b]indole isomer. This mixture solution in 150 ml ethanol was mixed with the equimolar amount of the (−)-dibenzoyltartaric acid. After some time the crystals of the salt enriched in (−)-trans-isomer precipitate.

The precipitate was several times re-crystallized from ethanol to achieve the resulting angle of rotation for free base [α]_{D}²⁰-58° (Cl, CHCl₃), for hydrochloride [α]²⁰_{D}: −10° (Cl, H₂O), as measured by Perkin-Elmer 241 polarimeter.

NMR spectors for optical isomers were recorded on the Varian-400 VXR with working frequency of 400 MHz (¹H NMR) and 100.6 MHz (¹³C NMR). Chemical shifts were measured using remaining solvents as internal standards.

The obtained (+)-trans-isomer and its dihydrochloric salt characteristics are:
free base: brutto formula: C₁₃H₁₇N₂; rotation angle [α]²⁰_{D}+58° (Cl, CHCl₃);
dihydrochloride: brutto formula C₁₃H₈N₂.2HCl; rotation angle [α]²⁰_{D}+7° (Cl, H₂O). Molecular weight is 276.1.
   The obtained (−)-trans-isomer and its dihydrochloric salt characteristics are:
free base: brutto formula: C₁₃H₁₇N₂; rotation angle [α]²⁰_{D}−58° (Cl, CHCl₃);
dihydrochloride: brutto formula C₁₃H₁₈N₂.2HCl; rotation angle [α]²⁰_{D}−10′ (Cl, H₂O).

Molecular weight is 276.1.

The MNR spectra of the (+)- and (−)-trans-isomers coincide:

¹H NMR spectra (CDCl₃): 1.92 (multiplet, 2H); 2.08 (multiplet, 2H); 2.19 (singlet, 3H); 2.34 (singlet, 3H); 2.80 (double triplet, 1H); 2.94 (multiplet, 2H); 3.37 (double duplet, 1H); 3.89 (wide singlet, 1H); 6.58 (double duplet, 1H); 6.78 (singlet, 1H); 6.79 (duplet, 1H) m.d.;

¹³C NMR spectra: 20.76; 30.98; 45.83; 47.35; 54.27; 57.02; 67.98; 110.25; 122.77; 127.36; 128.63; 131.07; 149.10 m.d.

B) Evaluation of biological activity of the individual optical isomers (+)- and (−)-trans-2,8-dimethyl-2,3,4,4a,5,9b-hexahydro-1H-pyrido[4,3-b]indole.

Expected pharmacological properties of compounds being (+) and (−)-trans-2,8-dimethyl-2,3,4,4a,5,9b-hexahydro-1H-pyrido[4,3-b]indole isomers were evaluated individually.

Preclinical evaluation of nootropic and sedative activity of the compounds was performed in mice using spontaneous orientation (patroling behavior) test in the cross maze, that allows simultaneous evaluation of nootropic, tranquilizing, sedative and psychostimulating effects of the compounds.

For evaluation of the exploratory behavior of the animals the closed cross maze test was used (Salimov R. M., Evaluation of the ordering of the way in the process of exploratory behavior in mice. Journal of the Higher Nervous Activity, 1988, v. 38, #3, pp. 569-571).

The mouse was placed in the central compartment of the maze and the sequence of its visits in different compartments was recorded semi-automatically.

Test was completed after 12 visits (Salimov R. M., Evaluation of the ordering of the way in the process of exploratory behavior in mice. Journal of the Higher Nervous Activity, 1988, v.38, #3, pp. 569-571; Salimov R. M., McBride W. J., Sinclair J. D., Lumeng L., Li T. -K. Performance in the cross-maze and slip funnel tests of four pairs of rat lines selectively bred for divergent alcohol drinking behavior. Addict. Biol. 1996a, 1:273-280; Salimov, R. M., McBride, W. J., McKenzie, D. L., Lumeng, L. and Li, T. -K. Ethanol consumption by adolescent alcohol-preferring P rats on subsequent behavioural performance in the cross-maze and slip funnel tests. Alcohol. 1996b, 13: c. 297-300).

Subsequent computerized analysis of the record singled out a number of behavioral characteristics. The most informative among them were:

1) The total time spent by the animal in the central compartment of the maze—T_ChTm, and the total time spent by the animal in the side arms of the maze—T_GlTm.

These variables reflect the level of the motor activity of the animal and also reflect the intensity of their exploratory drive for the new environment. They can be used for the evaluation of the stimulating/thymoleptic or, on the contrary, sedative effect of the drugs (Salimov, R. M., McBride, W. J., McKenzie, D. L., Lumeng, L. and Li, T. -K. Ethanol consumption by adolescent alcohol-preferring P rats on subsequent behavioural performance in the cross-maze and slip funnel tests. Alcohol. 1996b, 13: 297-300); Salimov, R. M.; Salimova, N. B.; Shvets, L. N.; Maisky, A. I. Haloperidol administered subchronically reduces the alcohol-deprivation effect in mice. Alcohol 20:61-68; 2000; Markina N. V., Salimov R. M., Poletaeva I. I. Exploratory behaviour of F2 crosses of mouse lines having selected for different brain weight: A multivariate analysis. Prog. Neuro-Psychopharmacol. Biol. Psychiat. 2004, 28(3):583-589).

2) The latency (variable F_ChTm) and the length of the first arm visit (F_GlTm).

These variables reflect the level of anxiety of the animal in the new environment and can be used for the evaluation of the tranquilizing (anxiolythic) effect of the drug (Salimov, R. M. Different behavioral patterns related to alcohol use in rodents: A factor analysis. Alcohol. 1999, 17:157-162).

These parameters negatively correlate with the duration of the animal's stay in the open arms in the well-known elevated cross-maze test, that allows to utilise the selective changes in these parameters as anxiety characteristics, and to use them for the evaluation of the tranquilizing activity of the drugs (Salimov R. M., Markina N. B., Perepyolkina O. V., Maiskiy A. I., Poletaeva I. I. Quick tolerance to ethanol and voluntary consumption of high doses of ethanol in mice, selected by the brain weight. Journal of the Higher Nervous Activity, 2003, v.53, #1, pp. 100-106 Salimov R.; Salimova N.; Shvets L.; Shvets N. Effect of chronic piracetam on age-related changes of cross-maze exploration in mice. Pharmacol.Biochem.Behav. 1995, 52:637-640; Salimov R. M., McBride W. J., Sinclair J. D., Lumeng L., Li T. -K. Performance in the cross-maze and slip funnel tests of four pairs of rat lines selectively bred for divergent alcohol drinking behavior. Addict. Biol. 1996a, 1:273-280; Salimov, R. M., McBride, W. J., McKenzie, D. L., Lumeng, L. and Li, T. -K. Ethanol consumption by adolescent alcohol-preferring P rats on subsequent behavioural performance in the cross-maze and slip funnel tests. Alcohol. 1996b, 13: 297-300; Salimov, R. M. Different behavioral patterns related to alcohol use in rodents: A factor analysis. Alcohol. 1999, 17:157-162.; Markina N. V., Popova N. V., Salimov R. M., Salimova N. B., Savchuk N. B., Poletaeva I. I. Comparison of the anxiety level and stress-reactivity in mice, selected by the high and low brain weight. Journal of the Higher Nervous Activity, 1999, v.49, #5, pp. 789-798).

3) The duration of the first patroling cycle—variable F_PtrN, and the duration of the second patrolling cycle—variable S_PtrN.

These variables are measured by the number of the animal's visits in the maze arms, performed by the animal for the exploration of the environment before it visits all 4 arms at least once (Salimov R. M., Evaluation of the ordering of the way in the process of exploratory behavior in mice. Journal of the Higher Nervous Activity, 1988, v.38, #3, pp. 569-571; Salimov R. M., McBride W. J., Sinclair J. D., Lumeng L., Li T. -K. Performance in the cross-maze and slip funnel tests of four pairs of rat lines selectively bred for divergent alcohol drinking behavior.

Addict. Biol. 1996a, 1:273-280; Salimov, R. M., McBride, W. J., McKenzie, D. L., Lumeng, L. and Li, T. -K. Ethanol consumption by adolescent alcohol-preferring P rats on subsequent behavioural performance in the cross-maze and slip funnel tests. Alcohol. 1996b, 13: 297-300; Markina N. V., Salimov R. M., Poletaeva I. I. Exploratory behaviour of F2 crosses of mouse lines having selected for different brain weight: A multivariate analysis. Prog. Neuro-Psychopharmacol. Biol. Psychiat. 2004, 28(3):583-589). The more visits the animal needs to visit all four arms of the maze, that is to complete one cycle of patroling, the less “systematic” an efficient is the exploration of the maze.

4) The number of patrolling cycles—variable PatriN, executed during the time of the experiment, as one of the characteristics of the efficiency of exploratory behavior. The more patroling cycles were accomplished, the more “systematic” and efficient was the exploration of the maze.

Variables from groups 3 and 4 can be used for the evaluation of the nootropic activity of the drugs.

Patroling behavior as evaluated by variables described in groups 3 and 4 is selectively disrupted by specific factors that damage the brain, such as ionizing radiation (Grigoriev A. Yu., Salimov R. M. Behavioral criteria for prediction of the cerebral form of the ionizing radiation damage prognosis in rats. Radiobiology, 1988,v.28, #2, pp. 270-272), alcohol intoxication (Salimov R. M., Markina N. B., Perepyolkina O. V., Maiskiy A. I., Poletaeva I. I. Quick tolerance to ethanol and voluntary consumption of high doses of ethanol in mice, selected by the brain weight. Journal of the Higher Nervous Activity, 2003, v.53, #1, pp. 100-106), experimental neurotoxic brain damage (Salimov R. M., Salimova N. B. L-glutamate abolishes differential responses to alcohol deprivation in mice. Alcohol. 1993, 10:251-257) and aging (Salimov R.; Salimova N.; Shvets L.; Shvets N. Effect of chronic piracetam on age-related changes of cross-maze exploration in mice. Pharmacol.Biochem.Behav. 1995, 52:637-640). It is improved by nootropic drugs, such as Piracetam and Meclofenaxate (Salimov R. M., Evaluation of the ordering of the way in the process of exploratory behavior in mice. Journal of the Higher Nervous Activity, 1988, v.38, #3, pp. 569-571; Salimov R.; Salimova N.; Shvets L.; Shvets N. Effect of chronic piracetam on age-related changes of cross-maze exploration in mice. Pharmacol.Biochem.Behav. 1995, 52:637-640; Salimov R. M., Kovalev G. I. Effects of N— cholinergic drugs on behavior in the explorative cross-maze in mice: comparison with cognitive enhancers. Eur. Neuropsychopharmacology, 2005, v.15, Suppl. 2, S 230).

5) The number of right turns—variable R_TrnN, and the number of left turns—variable L_TrN, executed by the animals moving from one arm of the maze to the other, as well as asymmetry index—variable rl_Ind—of these turns:

rl_Ind ═R_TrnN*(R_TrnN+L_TrnN)⁻¹

6) The spontaneous stereotype behavior—variable S_VisN, that characterizes animal's visiting two arms of the maze in turn more than two times in a row.

A decrease in spontaneous stereotype behavior is typical for some sedative drugs, for example Haloperidol, and for some nootropic drugs, for example Piracetam.

In experiments male C57BL/6 mice were used. Mice were housed in vivarium with natural light cycle and on the standard food ration, in standard cages 3-5 mice in each. At the beginning of the experiments mice were 3-4 months old.

Control animals were injected physiological saline.

Animals in experimental groups were injected either saline solutions of the studied compounds, namely (+) or (−)-trans-2,3,4,4a,5,9b-hexahydro-2,8-dimethyl-1H-pyrido[4,3-b]indole individual isomers, or saline solutions of the known drugs Piracetam or Maklophenaxate. Animal behavior was evaluated 1 hour after the drug treatment. Drugs and compounds were administered into stomach in a starch suspension by an atraumatic needle.

Experiments were performed in the first half of the day between 10:00 AM and 3:00 PM in an isolated laboratory room, using a 70 dB “white noise” above the threshold of audibility of a human.

The data were processed using STATISTICA software and the Dispersion analysis with inbuilt function for the means comparison using Student's T-test.

### EXAMPLE 1

### Evaluation of the Sedative Effect of Haloperidol

Experimental animals were administered a single dose of neuroleptic Haloperidol (0.5 mg/kg), the processed data on the evaluation of animal's exploratory behavior in the cross-maze test are presented in Table 1.

TABLE 1
Sedative Effects of Haloperidol

| | |
|---|---|
| Control | Haloperidol |
| N = 15 | 1 mg/kg N = 15 |

| Variable | M | SEM | M | SEM |
|---|---|---|---|---|
| F_PtrN | 6.20 | 0.61 | 6.09 | 0.55 |
| S_PtrN | 4.86 | 0.34 | 5.20 | 0.36 |
| PatrlN | 1.90 | 0.23 | 1.91 | 0.09 |
| F_ChTm | 11.35 | 1.90 | 19.30* | 3.07 |
| F_GlTm | 12.42 | 1.25 | 31.85* | 8.54 |
| T_ChTm | 54.51 | 6.59 | 100.65* | 12.26 |
| T_GlTm | 115.72 | 10.24 | 284.67* | 28.09 |
| R_TrnN | 3.40 | 0.79 | 3.91 | 0.53 |
| L_TrnN | 4.30 | 0.84 | 3.36 | 0.56 |
| rl_Ind | 0.46 | 0.10 | 0.54 | 0.06 |
| S_VisN | 5.29 | 0.90 | 2.35* | 0.99 |

Statistically significant differences from control (p < 0.05) are marked with *

Single administration of neuroleptic Haloperidol 0.5 mg/kg had a sedative effect as reflected by statistically significant reduction in the speed of animal's movement in the maze. Time spent by animals in the center and in the arms of the maze increased—increase in variables T_ChTm and T_GlTm. There is also a decrease in the spontaneous stereotype behavior (variable S_VisN). These observations are in agreement with earlier published data when this test was used (Salimov R. M., 1988; Salimov et al., 2000).

### EXAMPLE 2

### Evaluation of the Nootropic Effects of Piracetam and Maclophenaxate

Experimental animals were administered a single dose of well known nootropic drugs Piracetam (300 mg/kg) and Maclophenaxate (100 mg/kg), the processed data on the evaluation of animal's exploratory behavior in the cross-maze test is presented in Tables 2a and 2b.

TABLE 2a
Nootropic effects of Piracetam

| | |
|---|---|
| Control, | Piracetam, |
| N = 15 | N = 15 |

| Variable | M | SEM | M | SEM |
|---|---|---|---|---|
| F_PtrN | 5.9 | 0.4 | 4.5* | 0.2 |
| S_PtrN | 4.8 | 0.4 | 5.4 | 0.4 |
| PatrlN | 1.9 | 0.1 | 2.1 | 0.1 |
| F_ChTm | 17.7 | 3.0 | 11.6 | 2.7 |
| F_GlTm | 19.9 | 1.9 | 25.0 | 2.9 |
| T_ChTm | 45.2 | 2.7 | 46.1 | 3.6 |
| T_GlTm | 148.4 | 9.4 | 135.1 | 6.1 |
| R_TrnN | 5.2 | 0.5 | 3.4* | 0.5 |
| L_TrnN | 2.9 | 0.4 | 4.5* | 0.5 |
| F_PasN | 3.7 | 0.4 | 4.1 | 0.2 |
| rl_Ind | 0.6 | 0.0 | 0.4* | 0.1 |
| S_VisN | 6.9 | 0.8 | 4.3* | 0.8 |

Statistically significant differences from control by Student test (p < 0.05) are marked with *

TABLE 2b
Nootropic effects of Meclofenaxate

| | |
|---|---|
| Control, | Meclofenaxate, |
| N = 15 | N = 15 |

| | M | SEM | M | SEM |
|---|---|---|---|---|
| F_PtrN | 5.9 | 0.5 | 4.8* | 0.4 |
| S_PtrN | 5.5 | 0.2 | 4.6* | 0.2 |
| PatrlN | 2.0 | 0.2 | 2.5* | 0.1 |
| F_ChTm | 15.4 | 2.7 | 15.4 | 3.6 |
| F_GlTm | 22.7 | 3.9 | 29.1 | 5.5 |
| T_ChTm | 41.9 | 2.0 | 40.5 | 3.6 |
| T_GlTm | 131.2 | 10.3 | 145.4 | 8.0 |
| R_TrnN | 3.5 | 0.3 | 3.8 | 0.8 |
| L_TrnN | 4.9 | 0.4 | 4.5 | 0.7 |
| F_PasN | 3.5 | 0.3 | 3.6 | 0.5 |
| rl_Ind | 0.4 | 0.0 | 0.4 | 0.1 |
| S_VisN | 5.5 | 0.8 | 3.7 | 0.7 |

Statistically significant differences from control by Student test (p < 0.05) are marked with *

Single administration into mice's stomach of known nootropic drugs Piracetam (300 mg/kg) and Meclofenaxate (100 mg/kg) had a nootropic effect as reflected by statistically significant reduction in the duration of the first and second patrolling episodes—decrease in F_PtrN and S_PtrN values, as well as by the increase in the total number of patrolling behaviors—increase in the PatrIN value. These observations are in agreement with earlier published data when this test was used (Salimov R. M., 1988; Salimov R. M., et al., 1995; Salimov R. M., Kovalev G. I., 2005).

### EXAMPLE 3

### Evaluation of Sedative and Nootropic Effects of the (+)-trans-2,8-dimethyl-2,3,4,4a,5,9b-hexahydro-1H-pyrido[4,3-b]indole optic isomer According to the Invention

For evaluation was used individual (+)-trans-2,8-dimethyl-2,3,4,4a,5,9b-hexahydro-1H-pyrido[4,3-b]indole optic isomer obtained according to the invention.

Experimental animals were administered a single dose of (+)-trans-2,8-dimethyl-2,3,4,4a,5,9b-hexahydro-1H-pyrido[4,3-b]indole optic isomer (2.5 and 5.0 mg/kg), the processed data on the evaluation of animal's exploratory behavior in the cross-maze test is presented in Table 3.

TABLE 3
Effects of (+)-trans-2,8-dimethyl-2,3,4,4a,5,9b-hexahydro-1H-
pyrido[4,3-b]indole optic isomer on mice behavior in cross-maze test.

| | | | Trans-(+)-isomer, | | Trans-(+)-isomer, | |
|---|---|---|---|---|---|---|
| | Control, | | 2.5 mg/kg, | | 5.0 mg/kg, | |
| | N = 12 | | N = 12 | | N = 12 | |
| Variable | M | SEM | M | SEM | M | SEM |
| F_PtrN | 5.92 | 0.48 | 5.75 | 0.54 | 6.30 | 0.91 |
| S_PtrN | 6.11 | 0.48 | 4.55*⁺ | 0.25 | 5.43⁺ | 0.37 |
| PatrlN | 1.75 | 0.13 | 2.00 | 0.12 | 1.64 | 0.24 |
| F_ChTm | 7.49 | 2.32 | 9.18 | 2.86 | 24.60* | 6.44 |
| F_GlTm | 19.03 | 2.81 | 22.91 | 3.59 | 43.48* | 12.61 |
| T_ChTm | 30.13 | 1.40 | 53.27* | 5.19 | 58.27* | 9.89 |
| T_GlTm | 127.03 | 11.03 | 271.98*⁺ | 42.72 | 391.25*⁺ | 66.30 |
| R_TrnN | 2.83 | 0.42 | 2.67 | 0.73 | 3.09 | 0.69 |
| L_TrnN | 3.25 | 0.63 | 4.17 | 0.75 | 4.82 | 0.57 |
| rl_Ind | 0.48 | 0..08 | 0.39 | 0.09 | 0.38 | 0.08 |
| S_VisN | 6.67 | 0.63 | 5.75 | 0.95 | 5.55 | 0.88 |

*Statistically significant difference from control by Student t-test (p < 0.05)
⁺statistically significant difference as shown by Fisher criteria, Dispersion analysis (p < 0.05).

The data of tests indicate that single administration into mice's stomach of (+)-trans-2,8-dimethyl-2,3,4,4a,5,9b-hexahydro-1H-pyrido[4,3-b]indole optic isomer in 2.5 and 5.0 mg/kg doses had a sedative effect as reflected by statistically significant reduction in the speed of animal's movement in the maze. Time spent by animals in the center and in the arms of the maze increased—increase in variables T_ChTm and T_GlTm. These effects of (+)-trans-2,8-dimethyl-2,3,4,4a,5,9b-hexahydro-1H-pyrido[4,3-b]indole optic isomer are similar to the sedative effects of Haloperidol shown in Example 1 (Table 1).

Besides, (+)-trans-2,8-dimethyl-2,3,4,4a,5,9b-hexahydro-1H-pyrido[4,3-b]indole optic isomer in 2.5 and 5.0 mg/kg doses had a nootropic effect as reflected by statistically significant improvement in the patrolling behavior—decrease in S_PtrN values Table 3). This effect of (+)-trans-2,8-dimethyl-2,3,4,4a,5,9b-hexahydro-1H-pyrido[4,3-b]indole is similar to the effects of known nootropic drugs Piracetam and Meclofenaxate shown in Example 2 (Tables 2a and 2b).

### EXAMPLE 4

### Evaluation of Sedative and Nootropic Effects of the (−)-trans-2,8-dimethyl-2,3,4,4a,5,9b-hexahydro-1H-pyrido[4,3-b]indole optic isomer According to the Invention

For evaluation was used individual (−)-trans-2,8-dimethyl-2,3,4,4a,5,9b-hexahydro-1H-pyrido[4,3-b]indole isomer obtained according to the invention.

Experimental animals were administered into stomach a single dose of (+) or (−)-trans-2,8-dimethyl-2,3,4,4a,5,9b-hexahydro-1H-pyrido[4,3-b]indole isomer (2.5 and 5.0 mg/kg), the processed data on the evaluation of animal's exploratory behavior in the cross-maze test are presented in Table 4.

The results of the experiments indicate that single administration of (−)-trans-2,8-dimethyl-2,3,4,4a,5,9b-hexahydro-1H-pyrido[4,3-b]indole optic isomer in 2.5 and 5.0 mg/kg doses had a sedative effect as reflected by statistically significant reduction in the speed of animal's movement in the maze. Time spent by animals in the center and in the arms of the maze increased—increase in variables T_ChTm and T_GlTm. There is also a decrease in the spontaneous stereotype behavior (variable S_VisN). These effects of (−)-trans-2,8-dimethyl-2,3,4,4a,5,9b-hexahydro-1H-pyrido[4,3-b]indole are similar to the effects of Haloperidol reported in Example 1 (Table 1).

TABLE 4
Effects of (−)-trans-2,8-dimethyl-2,3,4,4a,5,9b-hexahydro-1H-
pyrido[4,3-b]indole isomer on mice behavior in cross-maze test

| | | | Trans-(−)-isomer, | | Trans-(−)-isomer, | |
|---|---|---|---|---|---|---|
| | Control, | | 2.5 mg/kg, | | 5.0 mg/kg, | |
| | N = 12 | | N = 12 | | N = 12 | |
| Variable | M | SEM | M | SEM | M | SEM |
| F_PtrN | 5.92 | 0.48 | 6.10 | 0.62 | 5.42 | 0.48 |
| S_PtrN | 6.11 | 0.48 | 4.43*⁺ | 0.30 | 5.00⁺ | 0.33 |
| PatrlN | 1.75 | 0.13 | 1.67 | 0.31 | 2.08 | 0.19 |
| F_ChTm | 7.49 | 2.32 | 10.75 | 3.09 | 8.33 | 2.85 |
| F_GlTm | 19.03 | 2.81 | 20.27 | 2.06 | 33.62* | 7.48 |
| T_ChTm | 30.13 | 1.40 | 33.10 | 2.31 | 42.23* | 4.41 |
| T_GlTm | 127.03 | 11.03 | 166.78*⁺ | 18.50 | 238.16*⁺ | 31.13 |
| R_TrnN | 2.83 | 0.42 | 3.08 | 0.61 | 5.08* | 0.56 |
| L_TrnN | 3.25 | 0.63 | 3.00 | 0.54 | 3.83 | 0.68 |
| rl_Ind | 0.48 | 0.08 | 0.50 | 0.09 | 0.58 | 0.06 |
| S_VisN | 6.67 | 0.63 | 6.33 | 0.64 | 4.17* | 0.69 |

*Statistically significant difference from control by Student t-test (p < 0.05)
⁺statistically significant difference as shown by Fisher criteria, Dispersion analysis (p < 0.05).

Besides, (+)-trans-2,8-dimethyl-2,3,4,4a,5,9b-hexahydro-1H-pyrido[4,3-b]indole optic isomer in 2.5 and 5.0 mg/kg doses had a nootropic effect as reflected by statistically significant improvement in the patrolling behavior—decrease in S_PtrN values (Table 3). These data are similar to the effects of known nootropic drugs Piracetam and Meclofenaxate shown in Example 2 (Tables 2a and 2b).

The results of the tests indicate that single administration of (+)- and (−)-trans-2,8-dimethyl-2,3,4,4a,5,9b-hexahydro-1H-pyrido[4,3-b]indole isomers in 2.5 and 5.0 mg/kg doses had a sedative effect as reflected by statistically significant reduction in the speed of animal's movement in the maze. Time spent by animals in the center and in the arms of the maze increased—increase in variables T_ChTm and T_GlTm. These effects of (+)- and (−)-trans-2,8-dimethyl-2,3,4,4a,5,9b-hexahydro-1H-pyrido[4,3-b]indole isomers are similar to the effects of Haldol shown in Example 1 (Table 1).

Besides, (+)- and (−)-trans-2,8-dimethyl-2,3,4,4a,5,9b-hexahydro-1H-pyrido[4,3-b]indole optic isomers in 2.5 and 5.0 mg/kg doses had a nootropic effect as reflected by statistically significant improvement in the patrolling behavior—decrease in S_PtrN values (Table 3). These data are similar to the nootropic effects of known drugs Piracetam and Meclofenaxate Known in Example 2 (Tables 2a and 2b).

Data also revealed differences in pharmacological profiles of the studied (+)- and (−)-trans-2,8-dimethyl-2,3,4,4a,5,9b-hexahydro-1H-pyrido[4,3-b]indole optic isomers. The (+)-trans-2,8-dimethyl-2,3,4,4a,5,9b-hexahydro-1H-pyrido[4,3-b]indole optic isomer demonstrates higher sedative effect than the (−)-trans-2,8-dimethyl-2,3,4,4a,5,9b-hexahydro-1H-pyrido[4,3-b]indole optic isomer. At the same time the (−)-trans-2,8-dimethyl-2,3,4,4a,5,9b-hexahydro-1H-pyrido[4,3-b]indole optic isomer significantly decreased spontaneous steretype behavior and the (+)-trans-2,8-dimethyl-2,3,4,4a,5,9b-hexahydro-1H-pyrido[4,3-b]indole optic isomer did not demonstrate this ability.

Thus the separation of the racemic isomer of trans-2,8-dimethyl-2,3,4,4a,5,9b-hexahydro-1H-pyrido[4,3-b]indole by method according to the invention two (+)- and (−)-trans-2,8-dimethyl-2,3,4,4a,5,9b-hexahydro-1H-pyrido[4,3-b]indole isomers are obtained according to the invention, that are antipodes of each other and demonstrate different biological activity and consequently different therapeutic application, which allows more efficient use of these compounds for the treatment of the individual condition of patients. Obtained compounds can be successfully used in different pharmaceutical preparations with different content of the active ingredients in combination with different inactive ingredients.

### INDUSTRIAL APPLICABILITY

Optic isomers obtained according to the invention, in which technologically acceptable procedures are used may be successfully utilized, for example, in pharmaceutical preparations according to the invention that can be prepared using known techniques.

1. The optical isomer (+)-trans-2,3,4,4a,5,9b-hexahydro-2,8-dimethyl-1H-pyrido[4,3-b]indole with positive rotation, having a structural formula: 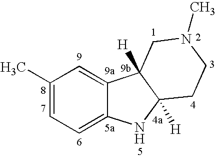
where hydrogen atoms in positions 4a and 9b are in trans-position.

2. The optical isomer (−)-trans-2,3,4,4a,5,9b-hexahydro-2,8-dimethyl -1H-pyrido[4,3-b]indole with negative rotation, having a structural formula: 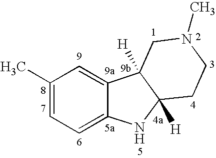
where hydrogen atoms in positions 4a and 9b are in trans-position.

3. The method of obtaining individual optical isomers of (+)-trans and (−)-trans-2,8-dimethyl-2,3,4,4a,5,9b-hexahydro-1H-pyrido[4,3-b]indole that utilizes interaction of 2,8-dimethyl-2,3,4,5-thetrahydro-1H-pyrido[4,3-b]indole with sodium borohydrate and with the etherate of trifluorine boron, treatment with hydrochloric acid, then alkilinization of the reaction mixture, separation of the trans-2,8-dimethyl-2,3,4,4a,5,9b-hexahydro-1H-pyrido[4,3-b]indole free base racemate, crystallization of its salts with (+)-dibenzoyltartaric acid from ethanol resulting in obtaining individual salts of the (+)-trans isomer From individual salt the (+)-trans-2,8-dimethyl-2,3,4,4a,5,9b-hexahydro-1H-pyrido[4,3-b]indole free base is obtained, the remainder is converted into the free racemic base of the trans-2,8-dimethyl-2,3,4,4a,5,9b-hexahydro-1H-pyrido[4,3-b]indole This base is crystallized with (−)-dibenzoyltartaric acid from ethanol resulting in obtaining individual salts of the (−)-trans isomer and later the (−)-trans-2,8-dimethyl-2,3,4,4a,5,9b-hexahydro-1H-pyrido[4,3-b]indole individual free base is obtained.

4. Pharmaceutical composition with nootropic and sedative activity, containing as an active ingredient optical isomer (+)-trans-2,3,4,4a,5,9b-hexahydro-2,8-dimethyl-1H-pyrido[4,3-b]indole and/or its pharmacologically acceptable salt in effective amount.

5. Pharmaceutical composition with nootropic and sedative activity, containing as an active ingredient optical isomer (−)-trans-2,3,4,4a,5,9b-hexahydro-2,8-dimethyl-1H-pyrido[4,3-b]indole and/or its pharmacologically acceptable salt in effective amount.
